(19) United States
(12) Patent Application Publication
(10) Pub. No.: US 2004/0209834 A1
(43) Pub. Date: Oct. 21, 2004

(54) Antagonists of the oncogenic activity of the MDM2, and use thereof in the treatment of cancers

(76) Inventors: Bruno Tocque, Courbevoie (FR); Bohdan Wasylyk, Illkirch (FR); Marie-Christine Dubs-Poterszman, Wolfisheim (FR)

Correspondence Address: WILEY, REIN & FIELDING, LLP, ATTN: PATENT ADMINISTRATION, 1776 K. STREET N.W., WASHINGTON, DC, 20006, US

(21) Appl. No.: 10/724,225

(22) Filed: Dec. 1, 2003

Related U.S. Application Data
(62) Division of application No. 09/029,327, filed on Mar. 23, 1998, now abandoned.
Application No. 09/029,327 is a 371 of international application No. PCT/FR96/01340, filed on Sep. 2, 1996.

(30) Foreign Application Priority Data
Sep. 4, 1995 (FR) FR95/10331

(51) Int. Cl. C12Q 1/68; G01N 33/574; A61K 48/00

(52) U.S. Cl. 514/44; 435/6; 435/7.23

(57) ABSTRACT

The present invention relates to the utilization of a compound capable of antagonizing at least partially the oncogenic activity of the protein Mdm2 for the preparation of a pharmaceutical composition intended more particularly to a treatment of cancers with no p53 context. It further relates to the viral vector comprising a nucleic acid sequence coding for a compound capable of inhibiting at least partially the oncogenic activity of the protein Mdm2, and to a corresponding pharmaceutical composition.

The present invention relates to a new method of treating hyperproliferative pathologies (cancers, restenoses, and the like) as well as to the corresponding pharmaceutical compositions.

It is now well established that a large majority of cancers is caused, at least in part, by genetic abnormalities which result either in the overexpression of one or more genes and/or the expression of one or more mutated or abnormal genes. For example, the expression of oncogenes generates a cancer in most cases. Oncogene is understood to mean a gene which is genetically affected and whose expression product disrupts the normal biological function of the cells, thus initiating a neoplastic state. A large number of oncogenes have so far been identified and partially characterized, such as especially the ras, myc, fos, erb, neu, raf, src, fms, jun and abl genes whose mutated forms appear to be responsible for a deregulation of cell proliferation.

In a normal cellular context, the proliferation of these oncogenes is probably checked, at least in part, by the generation of so-called tumour suppressor genes such as p53 and Rb. However, certain phenomena may come and disrupt this mechanism of cellular self-regulation and thereby promote the development of a neoplastic state. One of these events consists in mutations in the tumour suppressor genes. Accordingly, the form mutated by deletion and/or mutation of the p53 gene is involved in the development of most human cancers (Baker et al., Science 244 (1989) 217) and the inactivated forms of the Rb gene have been implicated in various tumours, and especially in retinoblastomas or in mesenchymatous cancers such as osteosarcomas.

The p53 protein is a nuclear phosphoprotein of 53 kD which is expressed in most normal tissues. It is involved in the control of the cell cycle (Mercer et al. Critic Rev. Eucar. Gene Express, 2, 251, 1992), transcriptional regulation (Fields et al., Sciences (1990) 249, 1046), replication of DNA (Wilcoq and Lane, (1991), Nature 349, 4290 and Bargonnetti et al., (1992) Cell 65 1083) and induction of apoptosis (Shaw et al., (1992) P.N.A.S. USA 89, 4495). Thus, any exposure of cells to agents capable, for example, of damaging the DNA thereof initiates a cascade of cellular signalling which results in a post-transcriptional modification of the p53 protein and in the transcriptional activation, by p53, of a number of genes such as gadd45 (growth arrest and DNA damage) (Kastan et al., Cell, 71, 587-597, 1992), p21 WAF/CIP (ElDeiry et al., Cancer Res., 54, 1169-1174, 1994) or alternatively mdm2 (mouse double minute) (Barak et al., EMBO J., 12, 461-468, 1993).

From the preceding text, it is clearly evident that the elucidation of the various biological functions of the range of proteins involved especially in this cell signalling pathway, of their modes of functioning and of their characteristics is of a major interest for the understanding of carcinogenesis and the development of effective therapeutic methods directed against cancer.

The present invention comes precisely within the framework of this context by reporting a new function of the Mdm2 protein.

The Mdm2 protein is a phosphoprotein with a molecular weight of 90 kD which is expressed from the mdm-2 gene (murine double minute 2). This mdm2 gene was originally cloned into a spontaneous tumour cell BALB/c 3T3 and it was observed that its overexpression greatly increases the tumoral power (Cahilly-Snyder et al., Somat. Cell. Mol. Genet., 13, 235-244, 1987; Fakharzadeh et al., EMBO J. 10, 1565-1569, 1991). An Mdm2/p53 complex has been identified in several cell lines containing both a wild-type p53 and mutated p53 proteins (Martinez et al., Genes Dev., 5, 151-159, 1991). In addition, it has been shown that Mdm2 inhibits the transcriptional activity of p53 on a promoter such as that of muscle creatine kinase indicating that Mdm2 may regulate the activity of p53 (Momand et al., Cell, 69, 1237-1245, 1992; Oliner et al., Nature, 362, 857-860, 1993).

In the light of all these results, the Mdm2 protein is therefore so far essentially recognized as a modulator of the activities of p53. By complexing the wild-type or mutated p53 proteins, it inhibits their transcriptional activity and contributes, in this manner, to the deregulation of cell proliferation. Consequently, the exploitation, at a therapeutic level, of this information consists mainly in searching for means of preventing this blockade of the p53 protein by Mdm2.

Unexpectedly, the applicant has demonstrated that this Mdm2 protein possessed an inherent oncogenic character, that is to say completely distinct from that associated with its form complexed with the p53 protein. More precisely, the Mdm2 protein develops oncogenic properties in a zero p53 context. In order to support this discovery, namely that the oncogenic properties of Mdm-2 are independent of p53 and in particular do not result from the inhibition of the transactivating activity of wild-type p53, we have shown that a mutant of p53 (p53 (14-19); Lin et al., Genes Dev., 1994, 8, 1235-1246) which conserved its transactivating properties but which no longer interacts with Mdm-2 is incapable of blocking the oncogenic properties of Mdm-2. It is also shown that Mdm-2 and in particular the 1-134 domain of Mdm-2 is capable of unblocking a stoppage of the cell cycle in G1 induced by the overexpression of p107. Mdm-2 therefore proves to be an important regulator of the factors involved in the control of the cell cycle, other than p53.

The present invention results, in part, from the demonstration that the protein sequence 1-134 of the sequence identified in SEQ ID No. 1, of the Mdm2 protein is sufficient to translate the oncogenic potential of the said protein.

It also results from the demonstration that it is possible to alter this oncogenic character of the Mdm2 protein using compounds capable of interacting with it. The present invention also describes particularly efficient systems allowing the in vivo delivery, directly into the tumours, of such compounds and thus the control of the development of cancers. The present invention thus offers a new approach which is particularly efficient for the treatment of tumours, in particular with a zero p53 context, such as the following cancers: colon adenocarcinomas, thyroid cancers, lung carcinomas, myeloid leukaemias, colorectal cancers, breast cancers, lung cancers, gastric cancers, oesophageal cancers, B lymphomas, ovarian cancers, cancers of the bladder, glioblastomas, and the like.

A first subject of the invention therefore consists in the use of a compound capable of antagonizing, at least partially, the oncogenic activity of the Mdm2 protein for the preparation of a pharmaceutical composition intended for the treatment of cancers with a zero p53 context.

For the purposes of the invention, cancer with a zero p53 context is understood to mean a cancer where p53 is thought to be incapable of exerting its tumour suppressor gene functions through any modification or any mechanism other than the attachment of Mdm-2 onto p53, this attachment preventing p53 from playing its role as tumour suppressor and allowing the cells to escape from a growth regulated by p53. There may be mentioned nonexhaustively, among these modifications or mechanisms blocking the tumour suppressor activity of p53, for example genetic alterations of the p53 gene (point mutations, deletions and the like), interaction with proteins other than Mdm-2, very rapid proteolytic degradation of the p53 protein linked to the presence of the E6 protein of high-risk human papillomaviruses such as HPV-16 and HPV-18, and the like.

For the purposes of the invention, the inhibition of the oncogenic activity of the Mdm2 protein may be achieved according to two methods.

It is preferably accomplished by acting directly at the level of the 1-134 domain thereof. Accordingly, any protein capable of binding to this domain will have an antagonistic role on the oncogenic properties of Mdm2.

However, this inhibitory effect may also be achieved via the interaction of a compound with a neighbouring domain, such as for example the 135-491 domain of mdm2, represented on the sequence SEQ ID No. 1 or its C-terminal sequence represented on the sequence SEQ ID No. 1. Consequently, the present invention relates, in addition, to the use of any compound which, although not directly interacting with this domain, is nevertheless capable of affecting the oncogenic character thereof.

According to a specific mode, the present invention relates to the use of a compound capable of binding at the level of the 1-134 domain of the sequence represented in SEQ ID No. 1 of the Mdm2 protein in order to prepare a pharmaceutical composition intended for the treatment of cancers with a zero p53 context.

As compound capable of interacting directly at the level of the 1-134 domain of the Mdm2 protein, there may be mentioned more particularly the scFV's directed specifically against this domain.

The ScFV's are molecules having binding properties comparable to those of an antibody and which are intracellularly active. They are more particularly molecules consisting of a peptide corresponding to the binding site of the variable region of the light chain of an antibody linked by a peptide linker to a peptide corresponding to the binding site of the variable region of the heavy chain of an antibody. It has been shown, by the applicant, that such ScFv's could be produced in vivo by gene transfer (Cf. application WO 94/29446).

They may also be peptides or proteins already known for their ability to bind specifically with the 1-134 domain of Mdm2, such as for example all or part of the binding domain of the p53 protein with SEQ ID No. 1 and more particularly all or part of one of the peptides 1-52, 1-41 and 6-41 of the p53 sequence represented in SEQ ID No. 2 (Oliner et al., Nature, 1993, 362, 857-860) or more simply all or part of the peptide 16-25 mapped more precisely (Lane et al., Phil. Trans. R. Soc. London B., 1995, 347 83-87), or even the peptides 18-23 of the human or murine p53's, or alternatively derived peptides close to those mentioned above in which the residues critical for the interaction with Mdm-2 will have been conserved (Picksley et al., Oncogene, 1994, 9., 2523-2529).

There may also be used, according to the invention, compounds [lacuna] of binding to domains close to the 1-134 domain of Mdm2 represented in SEQ ID No. 1 and affecting, by virtue of this binding, the oncogenic activity of the Mdm2 protein. In this capacity, there may be mentioned those interacting at the level of the C-terminal domain of the said protein, such as for example-the transcriptional factors TFII, TBP and TaF250 as well as the proteins interacting at the level of the 135-491 domain of Mdm2 represented in SEQ ID No. 1, such as for example the proteins L5 (ribosomal protein) and Rb (retinoblastoma protein) and the transcriptional factor E2F (regulated by Rb).

Another subject of the present invention also relates to the use of scFV's directed specifically against this 1-134 domain of the sequence represented in SEQ ID No. 1 of the Mdm2 protein in order to prepare a pharmaceutical composition intended for the treatment of cancers.

For the purposes of the invention, it is understood that all the interactions mentioned above substantially affect the oncogenic character of Mdm2. In addition, these proteins may be used, totally or in part, as long as use is made of their portion which is active in relation to one of the domains for binding with the Mdm2 protein and that this interaction leads to the oncogenic character of the latter being affected.

Within the framework of the present invention, these compounds may be used as they are or, advantageously, in the form of genetic constructs allowing their expression in vivo.

An advantageous specific embodiment of the present invention consists in using a nucleic sentence encoding a compound capable of antagonizing, at least partially, the oncogenic activity of the Mdm2 protein for the preparation of a pharmaceutical composition intended for the treatment of cancers with a zero p53 context.

In this perspective, the nucleic acids used within the framework of the invention may be of various types. They are preferably:

antisense nucleic acids,

oligoribonucleotides capable of directly binding one of the domains of the Mdm2 protein and of inhibiting its oncogenic activity (ligand oligonucleotide),

nucleic acids encoding, completely or in part, peptides or proteins capable of oligomerizing with one of the domains of Mdm2 and of inhibiting its oncogenic activity,

nucleic acids encoding intracellular antibodies (for example single-chain variable fragments derived from an antibody) directed against the 1-134 domain of the sequence SEQ ID No. 1 of the Mdm2 protein.

According to a specific embodiment of the present invention, the nucleic acid is an antisense nucleic acid. This antisense is a DNA encoding an RNA complementary to the nucleic acid encoding the Mdm2 protein and capable of blocking its transcription and/or its translation (antisense RNA) or a ribozyme.

More recently, a new type of nucleic acids capable of regulating the expression of target genes has been detected. These nucleic acids do not hybridize with the cellular mRNAs, but directly with the double-stranded genomic DNA. This new approach is based on the demonstration that some nucleic acids are capable of interacting specifically in the large groove of the DNA double helix to form locally triple helices, leading to an inhibition of the transcription of target genes. These nucleic acids selectively recognize the DNA double helix at the level of oligopurine.oligopyrimidine sequences, that is to say at the level of regions possessing an oligopurine sequence on one strand and an oligopyrimidine sequence on the complementary strand, and locally form thereon a triple helix. The bases of the third strand (the oligonucleotide) form hydrogen bonds (Hoogsteen or reverse Hoogsteen bonds) with the purines of the Watson-Crick base pairs. Such nucleic acids have especially been described by Prof. Helene in Anti-Cancer drug design 6 (1991) 569.

The antisense nucleic acids according to the present invention may be DNA sequences encoding antisense RNAs or ribozymes. The antisense RNAs thus produced may interact with an mRNA or a target genomic DNA and form with the latter double or triple helices. They may also be antisense sequences (oligonucleotides) optionally modified chemically, capable of interacting directly with the gene or the target RNA.

Still according to a preferred embodiment of the present invention, the nucleic acid is an antisense oligonucleoatide as defined above, optionally modified chemically. This may be in particular oligonucleotides whose phosphodiester backbone has been chemically modified, such as for example the oligonucleotide phosphonates, phosphotriesters, phosphoramidates and phosphorothioates which are described, for example, in patent application WO 94/08003. This may also be alpha-oligonucleotides or oligonucleotides conjugated with agents such as acrylating compounds.

For the purposes of the present invention, ligand oligonucleotide is understood to mean an oligoribonucleotide or an oligodeoxyribonucleotide capable of binding specifically to the Mdm-2 protein so as to inhibit its oncogenic function. Such nucleotides may, for example, be detected by “in vitro evolution” techniques such as for example the SELEX technique (Edgington, Bio/technology, 1992, 10, 137-140; patents U.S. Pat. No. 5,270,163 and WO 91/19813).

More generally, these nucleic acids may be of human, animal, plant, bacterial, viral or synthetic origin and the like. They may be obtained by any technique known to a person skilled in the art, and especially by screening of libraries, by chemical synthesis, or alternatively by mixed methods including the chemical or enzymatic modification of sequences obtained by screening of libraries.

As indicated later, they can, moreover, be incorporated into vectors such as plasmid, viral or chemical vectors. They can also be administered as they are, in naked DNA form according to the technique described in application WO 90/11092 or in a form complexed, for example, with DEAE-dextran (Pagano et al., J. Virol. 1 (1967) 891), with nuclear proteins (Kaneda et al., Science 243 (1989) 375), with lipids or cationic polymers (Felgner et al., PNAS 84 (1987)-7413), in the form of liposomes (Fraley et al., J. Biol. Chem. 255 (1980) 10431), and the like.

Preferably, the sequence used within the framework of the invention forms part of a vector. The use of such a vector indeed makes it possible to improve the administration of the nucleic acid into the cells to be treated, and also to increase its stability in the said cells, making it possible to obtain a lasting therapeutic effect. Furthermore, it is possible to introduce several nucleic acid sequences into the same vector, which also increases the efficiency of the treatment.

The vector used may be of various origins, as long as it is capable of transforming animal cells, preferably human cancer cells. In a preferred embodiment of the invention, a viral vector is used which may be chosen from adenoviruses, retroviruses, adeno-associated viruses (AAVs) or the herpesvirus.

In this regard, the subject of the present invention is also any viral vector comprising, inserted into its genome, a nucleic acid encoding a compound capable of antagonizing, at least partially, the oncogenic character of the Mdm2 protein.

More particularly, it relates to any recombinant virus comprising a nucleic acid sequence encoding a compound capable of binding to the Mdm2 protein so as to affect its oncogenic potential. In this context, the nucleic acid sequence may encode one of the peptides, proteins or transcriptional factors identified above.

More preferably, this nucleic acid sequence encodes an scFv or a peptide capable of interacting at the level of the 1-134 domain (SEQ ID No. 1) of the Mdm2 protein.

Advantageously, the viruses used within the framework of the invention are preferably defective, that is to say that they are incapable of autonomously replicating in the infected cell. Generally, the genome of the defective viruses used within the framework of the present invention therefore lacks at least the sequences necessary for the replication of the said virus in the infected cell. These regions maybe either removed (completely or in part), or made nonfunctional, or substituted by other sequences and especially by the sequence encoding the compound having an antagonistic role on the oncogenic properties of the Mdm2 protein. Preferably, the defective virus conserves nevertheless the sequences of its genome which are necessary for the encapsidation of the viral particles.

As regards more particularly adenoviruses, various serotypes, whose structure and properties vary somewhat, have been characterized. Among these serotypes, the use of the type 2 or 5 human adenoviruses (Ad 2 or Ad 5) or the adenoviruses of animal origin (see application FR 93 05954) is preferred within the framework of the present invention. Among the adenoviruses of animal origin which can be used within the framework of the present invention, there may be mentioned the adenoviruses of canine, bovine, murine (example: MAV1, Beard et al., Virology 75 (1990) 81), ovine, porcine, avian or alternatively simian (example: SAV) origin. Preferably, the adenovirus of animal origin is a canine adenovirus, more preferably a CAV2 adenovirus [Manhattan strain or A26/61 (ATCC VR-800) for example]. Preferably, adenoviruses of human or canine or mixed origin are used within the framework of the invention.

Preferably, the defective adenoviruses of the invention comprise the ITRs, a sequence allowing encapsidation and the sequence encoding the modulator of calpains. Still more preferably, in the genome of the adenoviruses of the invention, the E1 gene and at least one of the E2, E4, L1-L5 genes are nonfunctional. The viral gene considered may be made nonfunctional by any technique known to a person skilled in the art, and especially by total suppression, substitution, partial deletion or addition of one or more bases in the gene or genes considered. Such modifications may be obtained in vitro (on isolated DNA) or in situ, for example, by means of genetic engineering techniques, or alternatively by treatment by means of mutagenic agents.

The defective recombinant adenoviruses according to the invention can be prepared by any technique known to a person skilled in the art (Levrero et al., Gene 101 (1991) 195, EP 185 573; Graham, EMBO J. 3 (1984) 2917). In particular, they can be prepared by homologous recombination between an adenovirus and a plasmid carrying, inter alia, the DNA sequence encoding the ETS inhibitor. The homologous recombination occurs after co-transfection of the said adenoviruses and plasmid into an appropriate cell line. The cell line used should preferably (i) be transformable by the said elements, and (ii) contain the sequences capable of complementing the defective adenovirus genome part, preferably in integrated form in order to avoid the risks of recombination. By way of example of a line, there may be mentioned the human embryonic kidney line 293 (Graham et al., J. Gen. Virol. 36 (1977) 59) which contains especially, integrated into its genome, the left-hand part of the genome of an Ad5 adenovirus (12%). Strategies for the construction of vectors derived from adenoviruses have also been described in applications Nos. FR 93 05954 and FR 93 08596.

Next, the adenoviruses which have multiplied are recovered and purified according to conventional molecular biological techniques, as illustrated in the examples.

As regards the adeno-associated viruses (AAVs), they are relatively small DNA viruses which integrate into the genome of the cells which they infect, in a stable and site-specific manner. They are capable of infecting a broad spectrum of cells, without inducing any effect on cell growth, morphology or differentiation. Moreover, they do not seem to be involved in pathologies in man. The genome of the AAVs has been cloned, sequenced and characterized. It comprises about 4700 bases, and contains, at each end, an inverted repeat region (ITR) of about 145 bases, which serves as replication origin for the virus. The remainder of the genome is divided into 2 essential regions carrying the encapsidation functions: the left-hand part of the genome, which contains the rep gene involved in the viral replication and the expression of the viral genes; the right-hand part of the genome, which contains the cap gene encoding the virus capsid proteins.

The use of AAV-derived vectors for the transfer of genes in vitro and in vivo has been described in the literature (see especially WO 91/18088; WO 93/09239; U.S. Pat. No. 4,797,368, U.S. Pat. No. 5,139,941, EP 488 528). These applications describe various AAV-derived constructs in which the rep and/or cap genes are deleted and replaced by a gene of interest, and their use for the transfer in vitro (on cells in culture) or in vivo (directly in an organism) of the said gene of interest. The defective recombinant AAVs according to the invention can be prepared by co-transfection, into a cell line infected by a human helper virus (for example an adenovirus), of a plasmid containing the sequence encoding the ETS inhibitor bordered by two AAV inverted repeat regions (ITR), and of a plasmid carrying the AAV encapsidation genes (rep and cap genes). The recombinant AAVs produced are then purified by conventional techniques.

As regards the herpesviruses and the retroviruses, the construction of recombinant vectors has been widely described in the literature: see especially Breakfield et al., New Biologist 3 (1991) 203; EP 453242, EP 178220, Bernstein et al. Genet. Eng. 7 (1985) 235; McCormick, BioTechnology 3 (1985) 689, and the like. In particular, the retroviruses are integrative viruses which selectively infect dividing cells. They therefore constitute vectors of interest for cancer applications. The genome of the retroviruses essentially comprises two LTRs, an encapsidation sequence and three coding regions (gag, pol and env) In the recombinant vectors derived from retroviruses, the gag, pol and env genes are generally deleted, completely or in part, and replaced by a heterologous nucleic acid sequence of interest. These vectors can be prepared from various types of retrovirus such as especially MoMuLV (“murine moloney leukaemia virus”; also called MOMLV), MSV (“murine moloney sarcoma virus”), HaSV (“harvey sarcoma virus”); SNV (“spleen necrosis virus”); RSV (“rous sarcoma virus”) or alternatively Friend's virus.

To construct recombinant retroviruses comprising a sequence of interest, a plasmid comprising especially the LTRs, the encapsidation sequence and the said sequence of interest is generally constructed and then used to transfect a so-called encapsidation cell line capable of providing in trans the retroviral functions which are deficient in the plasmid. Generally, the encapsidation lines are therefore capable of expressing the gag, pol and env genes. Such encapsidation lines have been described in the prior art, and especially the PA317 line (U.S. Pat. No. 4,861,719); the PsiCRIP line (WO 90/02806) and the GP+envAm-12 line (WO 89/07150). Moreover, the recombinant retroviruses may contain modifications in the LTRs so as to suppress the transcriptional activity, as well as extended encapsidation sequences, comprising part of the gag gene (Bender et al., J. Virol. 61 (1987) 1639). The recombinant retroviruses produced are then purified by conventional techniques.

Advantageously, in the vectors of the invention, the sequence encoding the compound having antagonistic properties on the oncogenic character of Mdm2 is placed under the control of signals allowing its expression in tumour cells. Preferably, these are heterologous expression signals, that is to say signals different from those naturally responsible for the expression of the inhibitor. They may be in particular sequences responsible for the expression of other proteins, or of synthetic sequences. In particular, they may be promoter sequences of eukaryotic or viral genes. For example, they may be promoter sequences derived from the genome of the cell which it is desired to infect. Likewise, they may be promoter sequences derived from the genome of a virus, including the virus used. In this regard, there may be mentioned, for example, the E1A, MLP, CMV, RSV-LTR promoters and the like. In addition, these expression sequences may be modified by addition of activating or regulatory sequences or of sequences allowing a tissue-specific expression. It may, indeed, be particularly advantageous to use expression signals active specifically or predominantly in tumour cells so that the DNA sequence is expressed and produces its effect only when the virus has effectively infected a tumour cell.

In a specific embodiment, the invention relates to a defective recombinant virus comprising a cDNA sequence encoding a compound possessing antagonistic properties on the oncogenic character of Mdm2 under the control of a viral promoter, preferably chosen from RSV-LTR and the CMV promoter.

Still in a preferred mode, the invention relates to a defective recombinant virus comprising a DNA sequence encoding a compound possessing antagonistic properties on the oncogenic character of Mdm2 under the control of a promoter allowing predominant expression in tumour cells.

The expression is considered to be predominant for the purposes of the invention when, even if a residual expression is observed in other types of cells, the levels of expression are higher in tumour cells.

The present invention also extends to the use of a nucleic sequence encoding intracellular antibodies or alternatively scFV, which are directed against the 1-134 domain of the sequence of the Mdm2 protein represented in SEQ ID No. 1 for the preparation of a pharmaceutical composition intended in general for the treatment of cancer.

It also relates to any pharmaceutical composition comprising a compound capable of inhibiting the oncogenic activity of the Mdm2 protein, or a nucleic acid sequence encoding such a compound. According to a specific embodiment of the invention, this composition comprises one or more defective recombinant viruses as described above. These pharmaceutical compositions may be formulated for topical, oral, parenteral, intranasal, intravenous, intramuscular, subcutaneous, intraocular or transdermal administration and the like. Preferably, the pharmaceutical compositions of the invention contain a vehicle pharmaceutically acceptable for an injectable formulation, especially for a direct injection into the patient's tumour. This may be in particular isotonic sterile solutions or dry, especially freeze-dried, compositions which, upon addition, depending on the case, of sterilized water or of physiological saline, allow the preparation of injectable solutions. Direct injection into the patient's tumour is advantageous because it makes it possible to concentrate the therapeutic effect at the level of the affected tissues.

The doses of defective recombinant virus used for the injection may be adjusted according to various parameters, and especially according to the viral vector, the mode of administration used, the relevant pathology or alternatively the desired duration of treatment. In general, the recombinant adenoviruses according to the invention are formulated and administered in the form of doses of between 10⁴ and 10¹⁴ pfu/ml, preferably 10⁶ to 10¹⁰ pfu/ml. The term pfu (“plaque forming unit”) corresponds to the infectivity of a virus solution and is determined by infecting an appropriate cell culture and measuring, generally after 48 hours, the number of plaques of infected cells. The techniques for determining the pfu titre of a viral solution are well documented in the literature. As regards retroviruses, the compositions according to the invention may directly comprise the producing cells, for their implantation.

The pharmaceutical compositions according to the invention are particularly advantageous for neutralizing the oncogenic activity of the Mdm2 proteins and consequently for modulating the proliferation of certain cell types.

In particular, these pharmaceutical compositions are appropriate for the treatment of cancers possessing a zero p53 such as for example the following cancers: colon adenocarcinomas, thyroid cancers, lung cancers, myeloid leukaemias, colorectal cancers, breast cancers, lung cancers, gastric cancers, oesophageal cancers, B lymphomas, ovarian cancers, cancers of the bladder, glioblastomas and the like.

The present invention is advantageously used in vivo for the destruction of cells undergoing hyperproliferation (i.e. undergoing abnormal proliferation). It is thus applicable to the destruction of tumour cells or of the smooth muscle cells of the vascular wall (restenosis).

Other advantages of the present invention will emerge on reading the examples and figures which follow, which should be considered as illustrative and nonlimiting.

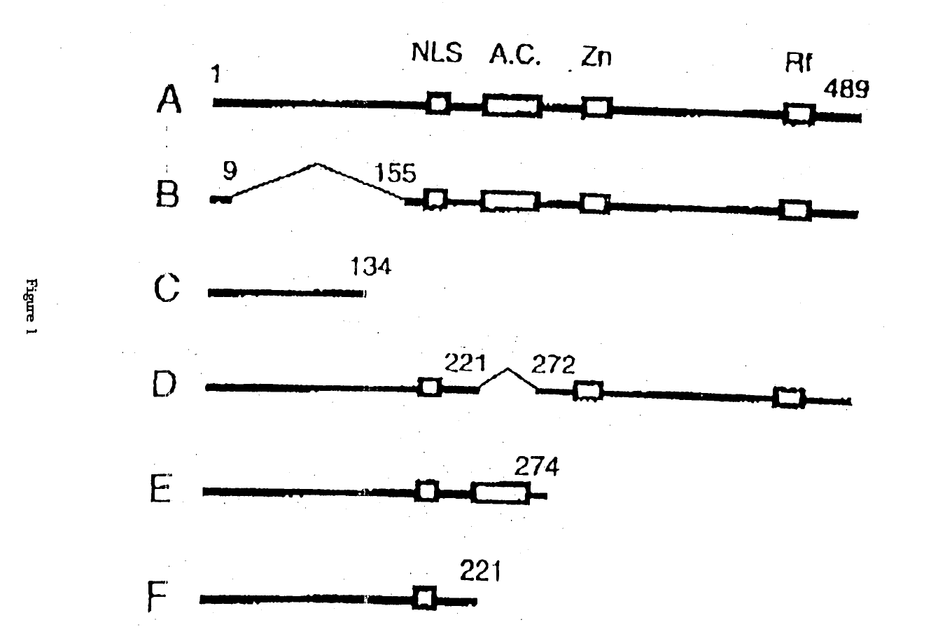

FIG. 1: Representation of the Mdm-2 proteins from A to F.

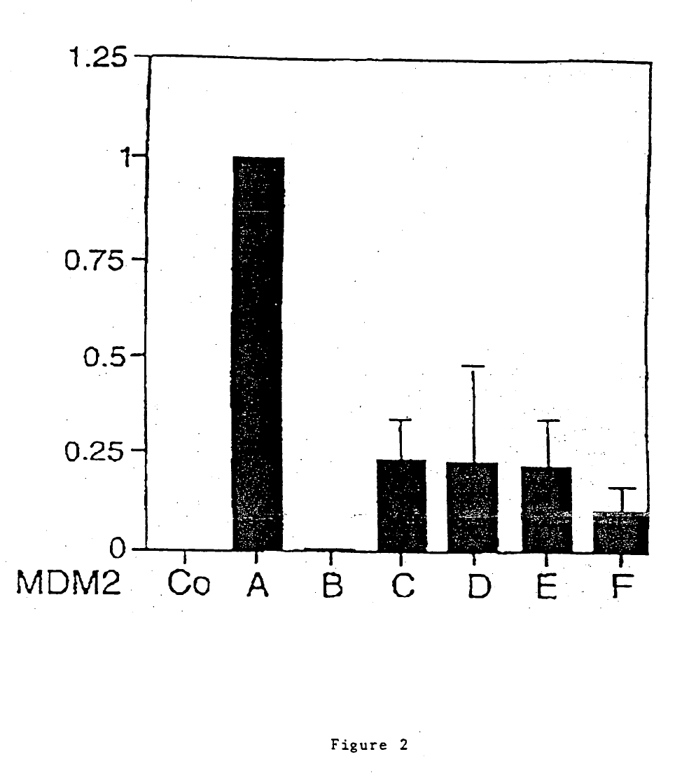

FIG. 2: Graph of the transfection of Saos-2 cells with plasmids expressing various Mdm-2 proteins

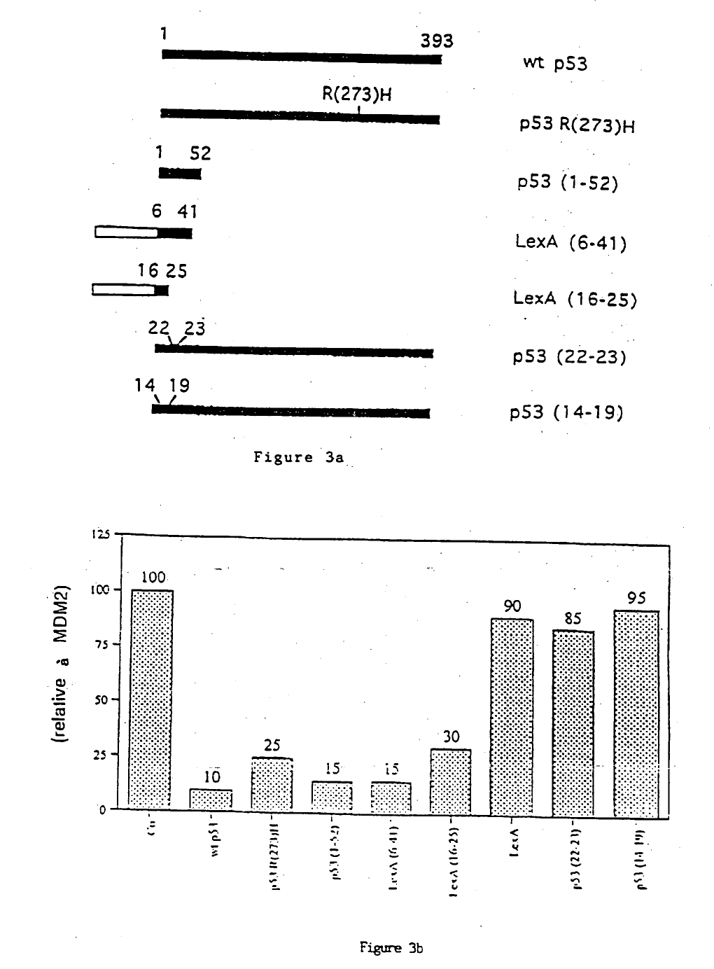

FIG. 3: Schematic representation of the inhibition of the transforming properties of Mdm2 by various p53's.

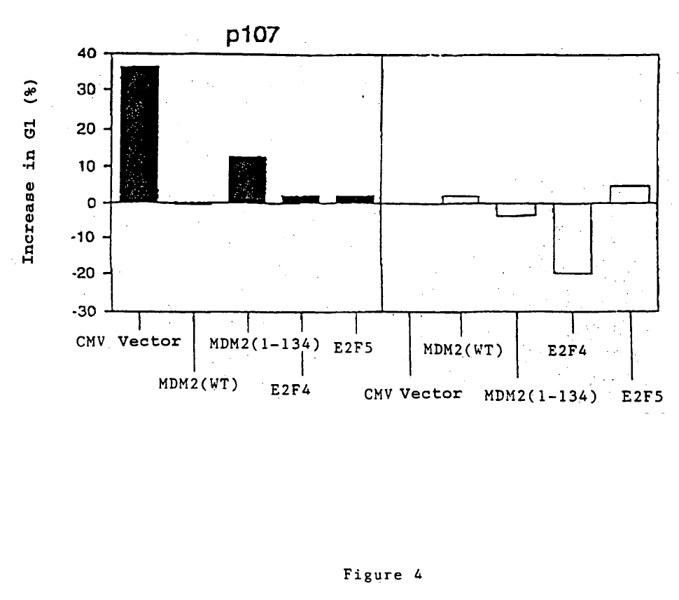

FIG. 4: Effect of an overexpression of Mdm2 on the cell cycle.

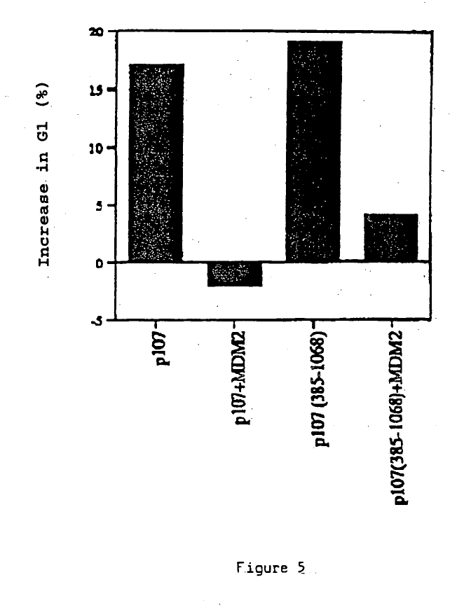

FIG. 5: Effect of an overexpression of Mdm2 on the cell cycle.

### GENERAL MOLECULAR BIOLOGY TECHNIQUES

The methods conventionally used in molecular biology such as preparative extractions of plasmid DNA, centrifugation of plasmid DNA in caesium chloride gradient, agarose or acrylamide gel electrophoresis, purification of DNA fragments by electroelution, phenol or phenol-chloroform extractions of proteins, precipitation of DNA in saline medium by ethanol or isopropanol, transformation in Escherichia coli, and the like are well known to a person skilled in the art and are abundantly described in the literature [Maniatis T. et al., “Molecular Cloning, a Laboratory Manual”, Cold Spring Harbor Laboratory, Cold Spring Harbor, N.Y., 1982; Ausubel F. M. et al. (eds), “Current Protocols in Molecular Biology”, John Wiley & Sons, New York, 1987].

For the ligations, the DNA fragments may be separated according to their size by agarose or acrylamide gel electrophoresis, extracted with phenol or with a phenol/chloroform mixture, precipitated with ethanol and then incubated in the presence of T4 phage DNA ligase (Biolabs) according to the supplier's recommendations.

The filling of the protruding 5′ ends may be performed by the Klenow fragment of DNA polymerase I of E. coli (Biolabs) according to the supplier's specifications. The destruction of the protruding 3′ ends is performed in the presence of T4 phage DNA polymerase (Biolabs) used according to the manufacturer's recommendations. The destruction of the protruding 5′ ends is performed by a controlled treatment with S1 nuclease.

The mutagenesis directed in vitro by synthetic oligodeoxynucleotides may be performed according to the method developed by Taylor et al. [Nucleic Acids Res. 13 (1985) 8749-8764] using the kit distributed by Amersham.

The enzymatic amplification of DNA fragments by the so-called PCR technique [Polymerase-catalyzed Chain Reaction, Saiki R. K. et al., Science 230 (1985) 1350-1354; Mullis K. B. et Faloona F. A., Meth. Enzym. 155 (1987) 335-350] may be performed using a “DNA thermal cycler” (Perkin Elmer Cetus) according to the manufacturer's specifications. The amplification of the genomic DNA is carried out more particularly under the following conditions: 5 minutes at 100° C., 30 cycles of one minute at 95° C., 2 minutes at 58° C. and then 3 minutes at 72° C. by means of appropriate probes. The amplification products are analysed by gel electrophoresis.

The verification of the nucleotide sequences may be performed by the method developed by Sanger et al. [Proc. Natl. Acad. Sci. USA, 74 (1977) 5463-5467] using the kit distributed by Amersham.

Materials and Methods:

1. Constructs Used:

the plasmid pBKCMV is marketed by. Stratagene and contains the neomycin resistance gene;

the plasmids pC53ClN3 and p53-4.2. N3 respectively encoding wild-type p53 and p53 R273H are from A. Levine (Hinds et al., Cell Growth and Diff. (1990), 1, 571);

the plasmid pBKp53 (R273H) contains the human p53 minigene. It was obtained from pC53-4.2 N3;

the plasmid pBKMdm2 was obtained by cloning into pBKCMV a coding cassette consisting of the untranslated region of the end of the sequence encoding β-globin followed by the sequence encoding mdm2;

the plasmid pGKhygro expresses the hygromycin resistance gene (Nature (1990) 348, 649-651);

the plasmid pCMVNeoBam allowing the expression of the neomycin resistance gene (Hinds et al., (1990) Cell. Growth and Diff., 1, 571-580);

the plasmids pCMVp107 and pCMVCD20 allowing the expression of the protein p107 and of the surface marker CD20 (Zhu et al., (1993) Genes and Development, 7, 1111-1125);

the plasmids pCMVE2F-4 and pCMVE2F-5 allowing the expression of the proteins E2F-4 and E2F-5 (Sardet et al., (1995) Proc. Natl. Acad. Sc., 92, 2403-2407);

the plasmids pLexA, pLexA(6-41), pLexA(16-25) allowing the expression of the domain for attachment to DNA of LexA (aa 1 to 87) free or fused in phase with p53(6-41) or p53(16-25). pLexA(6-41) and pLexA(16-25) were obtained from the plasmid pLexApolyII constructed at LGME (Strasbourg).

the plasmids for eukaryotic expression of p107: p107(385-1068), p107(1-781) and p107(781-1068 (Zhu et al., EMBO J. 14 (1995) 1904),

the plasmid pSGK1HAp107 allows the in vitro and in vivo expression of p.107. p107 is in the context of a Kozak sequence and the HP epitope is expressed in fusion at the C-terminal end of p107,

the plasmids pBC-MDM2 and pBC-MDM2(1-134) were obtained by cloning MDM2 and MDM2(1-134) into pBC (Chatton et al., Biotechniques 18 (1995) 142),

the plasmids pGex-MDM2 and pGex-MDM2(1-177) were obtained by cloning MDM2 and MDM2(1-177) into pGex.

2. Method:

The expression of p53 is determined by Western blotting on the whole cell extract with the aid of a monoclonal antibody D01.

The expression of the mRMA encoding the Mdm2 protein is estimated by semiquantitative RT-PCR.

The absence of contamination of DNA is checked by PCR.

### EXAMPLE 1

### Demonstration of the Transforming Properties of mdm2

Saos-2 cells are transfected with either a plasmid pBKMDM2, a control plasmid pBKp53 (R273H) or a negative p53 control plasmid pBKCMV, and then selected for resistance to Geneticin 418(G418).

In a first assay, clones are selected individually and propagated whereas in the other 2 assays, the clones not isolated are cultured in a soft agar medium.

For that, 10⁴ cells are inoculated in duplicate in 0.375% soft agar. After 24 hours, the total number of colonies with more than 50 cells as well as the number of cells by colony (size of the colonies) are determined. Each value given corresponds to a mean of four experiments carried out in duplicate. The results obtained are presented in Table I. The clones in assay No. 1 corresponding to mdm2 are identified under M1 to M6, those for p53 (R273H) under p53-1 to p53-6 and those for the control under Co1 to Co5).

As expected, Co1 and Co4 do not express the transfected mdm2 and Co 1-3 the p53 protein.

TABLE I

| GROWTH ON | |
|---|---|
| SOFT AGAR | |
| MEDIUM | |
| COLONIES | EXPRESSION |

| | | |
|---|---|---|
| (SIZE) | MDM2 | P53 |

EXPERIMENT 1
(clones isolated)

| | | | | | |
|---|---|---|---|---|---|
| MDM2 | M1 | 634 | (100-600) | + | ND |
| | M2 | 594 | (100-600) | ++ | ND |
| | M3 | 460 | (100-600) | + | ND |
| | M4 | 310 | (50-500) | ++ | ND |
| | M5 | 57 | (50-200) | +/− | ND |
| | M6 | 23 | (50) | + | ND |
| Control | Co1 | 97 | (50-200) | — | — |
| | Co2 | 68 | (50-200) | ND | — |
| | Co3 | 35 | (50-100) | ND | — |
| | Co4 | 11 | (50) | — | ND |
| | Co5 | 4 | (50) | ND | ND |
| p53R | p53-1 | 190 | (50-600) | ND | +++ |
| (273)H | p53-2 | 137 | (50-300) | ND | ++++ |
| | p53-3 | 88 | (50-200) | ND | +++ |
| | p53-4 | 53 | (50-200) | ND | +++ |
| | p53-5 | 47 | (50-200) | ND | ++ |
| | p53-6 | 38 | (50-100) | ND | + |
| MDM2 | M-P1 | 395 | (100-1000) | ++ | ND |

EXPERIMENT 2

| | | | | | |
|---|---|---|---|---|---|
| Control | Co-P1 | 21 | (50-200) | ND | ND |
| | Co-P2 | 18 | (50-200) | ND | ND |
| MDM2 | M-P1 | 255 | (50-300) | ND | ND |
| | M-P2 | 220 | (50-300) | ND | ND |

EXPERIMENT 3

| | | | | | |
|---|---|---|---|---|---|
| Control | CoP1 | 110 | (50-200) | ND | ND |
| | Co-P2 | 110 | (50-200) | ND | ND |
| | Co-P3 | 100 | (50-200) | ND | ND |
| | Co-P4 | 75 | (50-200) | ND | ND |

### EXAMPLE 2

### The N-Terminal Region of Mdm-2 (1-134) SEQ ID No. 1 is Necessary and Sufficient to Stimulate the Growth of the Saos-2 Cells in Soft Agar

Saos-2 cells are transfected either with plasmids pBKCMV which express both the neo resistance and the mdm-2 proteins from A to F described in FIG. 1, or an empty control plasmid PBKCMV, and then selected for resistance to G418. The surviving cells are combined, amplified and then tested for the formation of colonies in soft agar. The results of FIG. 2 are expressed in number of clones formed in soft agar relative to that with whole mdm-2 (A). These results are obtained from two independent experiments representative of transfection in which between 3 and 7 pools of different cells were tested, according to the construct. They show clearly that the N-terminal domain of mdm-2 possesses oncogenic properties. The most efficient construct corresponds to the whole protein.

### Example 3

### Reversion of the Oncogenic Properties of Mdm-2 by the Wild-Type p53, Mutants of p53 and Fragments of p53

A batch of Saos-2 cells transformed by Mdm-2 is co-transfected with the plasmid pGKhygro and either pC53C1N3 (p53) pC53-4.2N3 p53(R273H, p53 (1-52), pLexA (6-41), pLexA(16-25), pLexA, p53(L14Q,F19S), p53(L22Q,W23S), or pCMVNeoBam, and then selected for hygromycin resistance in the presence of G418. 100,000 cells from 3 to 5 independent pools of resistant cells are inoculated in duplicate in soft agar (0.375%). After 25 days of culture, the colonies containing at least 50 cells are counted. FIG. 3 presents the results of a representative experiment and gives a schematic representation of the various p53's tested to inhibit the transforming properties of Mdm-2. It emerges from this experiment that only the constructs allowing the expression of proteins capable of binding to the mdm-2 protein, in this case p53, p53 R273H, p53(1-52), LexA(6-41), LexA(16-25) inhibit the oncogenic properties of Mdm-2. On the other hand, the double mutants which were shown to have lost the capacity to bind to mdm-2 (Lin et al., Gene Dev., 1994, 8, 1235-1246) do not have an inhibitory effect. The fact that the mutant p53(14-19) which conserved the transactivating properties of the wild-type p53 does not inhibit transformation by Mdm-2 confirms that the oncogenic properties of Mdm-2 are independent of the inhibition by Mdm-2 of the transactivating properties of p53.

### EXAMPLE 4

### Mdm-2 Inhibits the Blocking in G1 of the Cell Cycle Induced by p107 in Saos-2 Cells

Saos-2 cells are co-transfected with three types of plasmids, (i) a plasmid for the expression of CD-20 (pCMVCD20, 2 μg, encoding the cell surface marker CD-20), (ii) a CMV type expression plasmid (9 μg) (cytomegalovirus promoter) without coding sequence or encoding Mdm-2 (PBKCMVMdm2), the 1-134 domain of Mdm-2 (PBKCMVMdm2(1-134)), E2F-4 or E2F-5 (pCMVE2F-4, pCMVE2F-5), and (iii) a vector for expression of p107 (pCMVp107, 9 μg). The cells are then treated for FACScan analysis as described by Zhu et al., (Gene Dev., 1993, 7, 1111-1125). The results of a representative experiment are presented in FIG. 4. It demonstrates clearly that in the absence of over expressed p107, the-expression of Mdm-2 or of-its 1-134 domain have no effect on the cell cycle. On the other hand, the expression of Mdm-2 and, efficiently, its 1-134 domain are capable of lifting the stoppage of the cell cycle in G1 induced by p107. This example demonstrates clearly that Mdm-2 is not only an inhibitor of the transactivating activity of p53 but also a positive regulator of the cell cycle capable of inhibiting factors involved in the control thereof.

In a similar experiment, Saos-2 cells are co-transfected with 1 μg of p107(385-1068), 8 μg of pCMVNeoBam, 1 μg of pXJMDM2, 8 μg of pXJ41 and 2 μg of pCMVCD20. The results of a representative experiment are indicated in FIG. 5. They show that the expression of MDM2-can lift the blockage in GI induced by p107 and by the deletion mutant p107(385-1068) which is capable of interacting with MDM2.

### EXAMPLE 5

### MDM2 Interacts In Vitro and In Vivo with p107

This example demonstrates a physical interaction between MDM2 and p107, in vitro and in vivo. These results are correlated with the MDM2 activity at the level of the cell cycle (Example 4).

5.1. In Vitro

In vitro S35-labelled p107 is brought into contact with the protein GST-MDM2 (vector pGex-MDM2) or GST-MDM2(1-177) (vector pgex-MDM2(1-177)) immobilized on glutathion sepharose beads. P107 bound to MDM2 is revealed after polyacrylamide gel by autoradiography. The results obtained are presented in Table II below.

5.2. In Vivo

Cos cells are cotransfected with a plasmid pBC-MDM2 or pBC-MDM2(1-134) which express a fusion protein GST-MDM2 or GST-MDM2(1-134), with a plasmid for expression of p107 or of a mutant of p107. The GST-MDM2-p107 protein complexes obtained from total cell extracts are isolated on glutathion sepharose beads and the p107 proteins are revealed by Western blotting with an anti-p107 polyclonal antibody (Santa Cruz p107-C18). The results obtained are presented in Table II below.

| | p107 | p107 | p107 |
|---|---|---|---|
| p107 | (385-1068) | (1-781) | (385-1068) |

In vivo

| | | | | |
|---|---|---|---|---|
| GST-MDM2 | + | + | + | + |
| GST-MDM2 (1-134) | + | nd | nd | nd |

In vitro

| | | | | |
|---|---|---|---|---|
| GST-MDM2 | + | nd | nd | nd |
| GST-MDM2 (1-177) | + | nd | nd | nd |

The results demonstrate that there is a protein-protein interaction between MDM2 and p107 in vitro, and also in the cell. The region of MDM2 which is necessary for cellular transformation (1-134) is the region which interacts with p107. This region has been localized as being situated more precisely in a part of the “pocket domain”, region “A” and the “spacer”.

1. A method of determining the mdm2-dependent growth activity of a cell, wherein the cell comprises an introduced nucleic acid encoding and expressing an mdm2 protein, the method comprising introducing one or more vectors for expressing a p53 polypeptide or fragment thereof into the cell, and measuring the growth of the cell, whereby a p53 polypeptide or fragment and exhibiting a reduced cell growth level compared to a control is capable of inhibiting a transforming property of mdm2.

2. The method of claim 1, wherein the nucleic acid encoding mdm2 comprises the sequence encoding from amino acid 1 to amino acid 134 of SEQ ID NO.: 1.

3. The method of claim 1, wherein the cell is a human cell.

4. The method of claim 1, wherein the cell is a tumor cell.

5. The method of claim 1, wherein the cell is a saos-2 cell.

6. A method of detecting protein-protein interaction with a mdm2 protein, comprising expressing a mdm2 protein in a mammalian cell from an introduced nucleic acid encoding an mdm2 protein, expressing in the cell a cell cycle regulatory protein from an introduced nucleic acid encoding it, and comparing the cell growth or proliferation with and without the expressed mdm2 protein, whereby a change in cell growth or proliferation is indicative of binding to mdm2.

7. The method of claim 6, wherein the nucleic acid encoding mdm2 comprises the sequence encoding from amino acid 1 to amino acid 134 of SEQ ID NO.: 1.

8. The method of claim 6, wherein the cell is a human cell.

9. The method of claim 6, wherein the cell is a tumor cell.

10. The method of claim 6, wherein the cell is a saos-2 cell.


SEQUENCE LISTING (4 sequences)

SEQ ID NO: 1 - DNA (other nucleic acid), 1476 bp; single-stranded, linear
  CDS 1..1473
  description: /desc = “Oligonucleotide”
atgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtaagtgaacattcaggtgattggttggatcaggattcagtttcagatcagtttagtgtagaatttgaagttgaatctctcgactcagaagattatagccttagtgaagaaggacaagaactctcagatgaagatgatgaggtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaatctagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttcccctag

SEQ ID NO: 2 - protein, 491 aa
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 3 - DNA (other nucleic acid), 1182 bp; single-stranded, linear
  CDS 1..1179
  description: /desc = “Oligonucleotide”
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 4 - protein, 393 aa [sequence omitted]